(19) United States
(12) Patent Application Publication
(10) Pub. No.: US 2024/0316150 A1
(43) Pub. Date: Sep. 26, 2024

(54) METHOD FOR PREVENTION OR TREATMENT OF DISEASES CAUSED BY PATHOGEN-ASSOCIATED MOLECULAR PATTERN

(71) Applicant: SQ BIOPHARMA INC., Chengdu (CN)

(72) Inventors: Yuan-Ping HAN, Los Angeles, CA (US); Yucheng Qi, Chengdu (CN)

(21) Appl. No.: 18/732,612

(22) Filed: Jun. 3, 2024

Related U.S. Application Data
(63) Continuation-in-part of application No. PCT/CN2022/093068, filed on May 16, 2022.

(30) Foreign Application Priority Data
Dec. 3, 2021 (CN) 202111465553.0

(51) Int. Cl. A61K 38/17; A61K 9/28; A61K 31/785; A61P 1/16; A61P 29/00

(52) U.S. Cl. CPC A61K 38/1729; A61K 9/2853; A61K 31/785; A61P 1/16; A61P 29/00

(57) ABSTRACT

A method for prevention or treatment of diseases caused by a pathogen-associated molecular pattern (PAMP) from intestinal microorganisms, including administering a subject in need thereof a cationic polymer. The enteric coated formulation of the cationic polymer is applied to sequestrate the PAMP produced by intestinal microorganisms, to alleviate the related diseases. The diseases subjected to the treatment is inflammatory disorder, metabolic syndrome, type-2 diabetes, alcohol related diseases, non-alcoholic steatohepatitis, obesity, tissue damage, fatty liver, cirrhosis, inflammatory bowel diseases, multiple tumors and cancers, organ failure, or sepsis.

### CROSS-REFERENCE TO RELATED APPLICATIONS

This application is a continuation-in-part of International Patent Application No. PCT/CN2022/093068 with an international filing date of May 16, 2022, designating the United States, now pending, further claims foreign priority benefits to Chinese Patent Application No. 202111465553.0 filed Dec. 3, 2021. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### BACKGROUND

The disclosure relates to the field of pharmaceutical technology, and more particularly, to a method for prevention or treatment of a disease caused by a pathogen-associated molecular pattern derived from intestinal microbes.

The gastrointestinal tract contains a large number of microbes, including bacteria, viruses, and fungi, mainly localized in large intestine. About 1-1.5 kg of gut microbiota maintain human physiological health in a symbiotic relationship. However, factors such as aging, infection, poor diet (e.g., high in fat and sugar), and xenobiotics from environmental pollution can disrupt the gut microbiota, causing dysbiosis exhibited as increased levels of opportunistic pathogens and reduced bacterial diversity.

After the death of gut microbes, various substances known as pathogen-associated molecular patterns (PAMPs) are released. PAMPs are conserved molecular structures on pathogens that can trigger pathogenic responses when they enter the host. Recognized by innate immune cells of the host, PAMPs initiate an immune response. PAMPs include lipopolysaccharides (LPS), peptidoglycan (PGN), mannose, bacterial DNA, lipoteichoic acid, viral double-stranded RNA, and glucans. PAMPs primarily bind to pattern recognition receptors (PRRs) on the host cells. PRRs, expressed mainly on innate immune cells, can recognize one or more PAMPs. Upon binding to PAMPs, PRRs activate intracellular signaling pathways that lead to the expression of pro-inflammatory cytokines and other immune checkpoint receptors, triggering various types of immune response. Toll-like receptors (TLRs), a type of PRR, recognize specific PAMPs and signal microbial infection to the host. PAMPs are crucial for pathogen recognition and immune response, linking them to various infectious diseases and metabolic disorders. For example, LPS from Gram-negative bacteria can cause severe inflammatory responses and septic shock, a primary cause of bacterial infections. Additionally, PAMP recognition by PRR is associated with autoimmune diseases and chronic inflammatory conditions, as abnormal immune responses may lead to autoimmunity.

Lipopolysaccharide (LPS) is a component of the outer membrane of Gram-negative bacteria, consisting of a lipid and a polysaccharide. Upon entering the body, LPS binds to Toll-like receptor 4 (TLR-4) on host cells, initiating protein phosphorylation and signaling cascades that trigger downstream gene expression. LPS can induce cells to express pro-inflammatory cytokines such as TNF-α and IL-1, promoting inflammation. Prolonged inflammation can damage tissue and promote chronic diseases such as insulin resistance, type-2 diabetes, inflammatory bowel diseases (IBD), and cancer growth and metastasis.

Therapeutic methods for treatment of endotoxin-related diseases are largely inefficient in many ways. First, antibiotic therapy can control infections, but extended application of antibiotics can kill symbiotic bacteria in the gut, which may consequently cause influx of endotoxin and other PAMP into the body. Second, specific antibodies as a therapeutic agent can be used to neutralize endotoxins in the blood. However, long-term use of antibodies may activate the immune system, which may cause endotoxin-related shock. Third, using specific antibody therapies to antagonize or neutralize the pro-inflammatory cytokines like TNF-α or IL-1 are effective in clinical practice, but long-term use of the antibodies may reduce the therapeutic efficacy and even cause allergic reactions. Fourth, supportive treatments such as extracorporeal liver circulation (ECL) (“artificial liver”), fluid resuscitation, electrolyte balance, and nutritional support can be used to lower blood endotoxin levels. Polymyxin B, a peptide antibiotic, inhibits Gram-negative bacterial infections through direct binding to LPS in high affinity, while it is reserved for treating multidrug-resistant (MDR) or extensively drug-resistant (XDR) infections due to its renal toxicity. Despite its critical role in pathogenesis of many diseases, effective and safe methods to neutralize endotoxin as therapeutics are largely unknown.

### SUMMARY

To solve the aforesaid problems, the disclosure provides a method for prevention or treatment of a disease caused by a pathogen-associated molecular pattern from intestinal microorganisms. The method comprises administering to a patient in need thereof a cationic polymer as active pharmaceutical ingredient (API).

In a class of this embodiment, the cationic polymer is a cationic copolymer comprising an amine group, or a cationic peptide, or a derivative thereof.

In a class of this embodiment, the cationic polymer is an organic polymer; and the amine group is in the form of a secondary amine, a tertiary amine, or a quaternary amine

In a class of this embodiment, the organic polymer is prepared by introducing a nitro group through nitration to an aliphatic or aromatic polymer, and reducing the aliphatic or aromatic polymer comprising the nitro group, to obtain the organic polymer.

In a class of this embodiment, the organic polymer is at least one selected from a group consisting of a polystyrene-based quaternary ammonium salt, diethylaminoethyl (DEAE)-cellulose, a polymyxin B crosslinked polymer, polylysine, and a derivative thereof.

In a class of this embodiment, the cationic polymer comprises cholestyramine, colestipol, colesevelam, or a combination thereof, and is used to sequestrate intestinal PAMPs, such as endotoxins, microbe-derived CpG-DNA and RNA fragments, and indole related acid.

In a class of this embodiment, the cationic polymer is formulated into enteric coated tablet or capsule via orally administration to sequestrate the PAMPs produced by intestinal microorganisms, thereby alleviating related diseases.

In a class of this embodiment, the diseases treated by the cationic polymer in formulation are inflammatory related disorders, such as insulin resistance, metabolic syndrome, type-2 diabetes, obesity, various tumors and cancers, of which are caused, in part, by the intestinal PAMPs.

In a class of this embodiment, the cationic peptide is at least one selected from a group consisting of poly-lysine, α-defensin-5, α-defensin-6, and structurally modified derivatives thereof.

In a class of this embodiment, the cationic peptide or the derivative thereof is formulated into enteric coated tablets or capsule for configuration of gut microbiota of the patient, thereby alleviating or treating the diseases.

In a class of this embodiment, the formulation of the cationic peptide or the derivative thereof is used to prevent or treat inflammatory diseases, such as metabolic syndrome, type-2 diabetes, obesity, fatty liver, cirrhosis, various tumors and cancers, and disorders from novel coronavirus infection (Sars-covid-2 virus), acute pneumonia and organ failure due to endotoxin or other PAMPs entering bloodstream from the gut.

In a class of this embodiment, the cationic polymer as an active pharmaceutical ingredient (API) is resistant to enzymatic degradation in digestive track and not absorbable by human subjects in therapeutic application.

In a class of this embodiment, the enteric coated formulation of the cationic polymer or the cationic peptide is administered orally.

In a class of this embodiment, the cationic polymer can bind and sequestrate the gut-microbe-derived PAMPs including lipopolysaccharides, short-chain fatty acids, intestinal hydrogen sulfide, indole sulfate, short-chain fatty acids, unmethylated DNA or RNA fragments, flagellin, and S protein and other acidic components from SARS-COV-2 in severe COVID-19 illness.

The disclosure is a method to utilize cationic polymer to sequestrate and excrete the PAMPs produced by intestinal microorganisms, for alleviating the PAMP related liver diseases, including but not limited to alcoholic hepatitis (AH), non-alcoholic steatohepatitis (NASH), drug induced hepatitis, autoimmune hepatitis such as primary biliary cholangitis (PBC) and primary sclerosing cholangitis (PSC), viral mediated hepatitis including hepatitis B virus (HBV) and hepatitis C virus (HCV), fulminating hepatitis, liver failure, hepatic fibrosis and cirrhosis.

### BRIEF DESCRIPTION OF THE DRAWINGS

FIG. 1A-1D demonstrates the ability of in vitro sequestration of lipopolysaccharides (LPS) by two types of the cationic polymer as active pharmaceutical ingredients (API); FIG. 1A shows the standard curve for measuring the concentration of LPS (endotoxin) in solution; FIG. 1B shows the remaining LPS levels in the supernatant after incubating of cholestyramine (Chol) with endotoxin and separating by centrifugation; FIG. 1C shows the ability of colesevelam (CV) to sequestrate indole-3-acetic acid; FIG. 1D shows the ability of colesevelam (CV) and activated charcoal to sequestrate endotoxins; according to Example 5 of the disclosure;

FIG. 2 shows the ability of reduction of lipopolysaccharides (LPS) levels in the bloodstream by oral administration of cholestyramine (Chol), while polystyrene (PS) is used as negative control, according to Example 6 of the disclosure;

FIG. 3 shows the capacity of balancing gut microbiota by oral administration of cholestyramine (Chol), according to Example 7 of the disclosure; where, “Firm” refers to Firmicutes, “Bact” refers to Bacteroidetes, “Gamma-proteo” refers to Gamma-proteobacteria, “Akk” refers to Akkermansia muciniphila, and “others” refers to other bacteria;

FIG. 4 presents the improved NAS scores for fatty liver by oral administration of cholestyramine (Chol), according to Example 8 of the disclosure;

FIG. 5 displays the distribution of CD3+ cells in the liver tissues by the mice under high fat feeding without vitamin D supplement, and improvement by oral administration of cholestyramine (Chol), according to Example 9 of the disclosure;

FIG. 6 shows the mRNA expression of TNF-α/RPL-19 in liver tissues by the mice under high fat feeding without vitamin D supplement, and improvement by oral administration of cholestyramine (Chol), according to Example 10 of the disclosure;

FIG. 7 shows the levels of alanine aminotransferase (ALT) in the bloodstream by the mice under high fat feeding without vitamin D supplement, and improvement by oral administration of cholestyramine (Chol), according to Example 11 of the disclosure;

FIG. 8 shows that endotoxin (LPS) exacerbates the drug-induced cirrhosis), measured as Sirius Red staining of the liver tissues, according to Example 12 of the disclosure;

FIG. 9 shows that endotoxin (LPS) exacerbates the drug-induced cirrhosis, measured as the mRNA expression of type-1 collagen, according to Example 13 of the disclosure;

FIG. 10 shows ability that endotoxin (LPS) exacerbates the drug-induced hepatic inflammation, measured as the mRNA expression of IL-1β in the liver under different experimental conditions, according to Example 14 of the disclosure;

FIG. 11 shows the mRNA expression of α-defensin-5 (DEFA5) produced by Paneth cells in ileal tissues under different experimental conditions, according to Example 15 of the disclosure;

FIG. 12 shows the mRNA expression of Muc-2 in ileal tissues under different experimental conditions according to Example 16 of the disclosure;

FIG. 13 shows the mRNA expression of Occludin in ileal tissues under different experimental conditions, according to Example 17 of the disclosure;

FIG. 14 presents the ability of reduction of liver fibrosis or cirrhosis by oral administration of cholestyramine (Chol), measured as Sirius Red staining of the liver tissues, according to Example 18 of the disclosure;

FIG. 15 shows the ability of reduction of liver fibrosis or cirrhosis by oral administration of cholestyramine (Chol), measured as mRNA expression of type-1 collagen in the liver tissues under different experimental conditions, according to Example 19 of the disclosure;

FIG. 16 demonstrates the ability of relieving liver injury by oral administration of cholestyramine (Chol), measured as levels of plasma transaminase (ALT) under different experimental conditions, according to Example 20 of the disclosure;

FIG. 17 indicates the ability of reduction of liver injury by oral administration of cholestyramine (Chol), measured as levels of plasma transaminase (AST) under different experimental conditions, according to Example 21 of the disclosure;

FIG. 18 illustrates the ability of reduction of blood levels of total bilirubin (TBIL) by oral administration of cholestyramine (Chol), under different experimental conditions, according to Example 22 of the disclosure;

FIG. 19 shows the ability of reduction of blood levels of direct bilirubin (DBIL) by oral administration of cholestyramine (Chol), under different experimental conditions, according to Example 23 of the disclosure;

FIG. 20 displays the activation of monocytes induced by spike protein (S protein from Sars-Covid-2) and the mRNA expression of pro-inflammatory cytokine IL-1β, according to Example 24 of the disclosure;

FIG. 21 illustrates the activation of monocytes induced by the spike protein (S protein from Sars-Covid-2) and the mRNA expression of pro-inflammatory cytokine TNF-α, according to Example 25 of the disclosure;

FIG. 22 show the synergistic effect of spike protein (S protein from Sars-Covid-2) and endotoxins in activating monocytes and the mRNA expression of pro-inflammatory cytokine IL-1β, according to Example 26 of the disclosure;

FIG. 23 shows the synergistic effect of spike protein (S protein from Sars-Covid-2) and endotoxins in activating monocytes and the mRNA expression of pro-inflammatory cytokine TNF-α, according to Example 27 of the disclosure;

FIG. 24 shows PAS staining of intestinal mucosa and lysozyme staining in mouse ileal tissues, as indicators for phagocytic activity of intestinal epithelial cells, and matrix metalloproteinase-7 (Mmp-7) staining as an indicator for α-defensin activation in Paneth cells, according to Example 28 of the disclosure;

FIG. 25 shows the mRNA expression of α-defensin-2 and α-defensin-5 in mouse ileal tissues, according to Example 29 of the disclosure;

FIG. 26A-26B show results that mice under hepatic injury can be ameliorated by oral administration of synthetic human α-defensin-5 (DEFA5), according to Example 30 of the disclosure; FIG. 26A shows that mice under hepatic injury are given oral administration of synthetic human α-defensin-5 (10 mg, twice a week) for an additional 8 weeks; FIG. 26B shows Sirius Red staining for the liver fibrosis and CD3+ staining of liver tissue, according to Example 30 of the disclosure.

FIG. 27 shows the results of intestinal permeability by the mice under liver injury and subjected to oral administration of synthetic human α-defensin-5 (DEFA5), according to Example 31 of the disclosure;

FIG. 28 shows the results of bloodstream levels of endotoxin by the mice under hepatic injury and were orally given synthetic human α-defensin-5 (DEFA5), according to Example 32 of the disclosure;

FIG. 29 shows the results of gut microbiota by the mice under liver injury and subjected to oral administration of synthetic human α-defensin-5 (DEFA5), according to Example 33 of the disclosure.

### DETAILED DESCRIPTION

To further illustrate the disclosure, embodiments detailing a method for prevention or treatment of a disease caused by a pathogen-associated molecular pattern (PAMP) from intestinal microorganisms are described below. It should be noted that the following embodiments are intended to describe and not to limit the disclosure.

The term “CpG oligodeoxynucleotides (CpG ODNs)” as used herein, refers to short single-stranded DNA molecules consisting of a cytosine deoxynucleotide (“C”) followed by a guanine deoxynucleotide (“G”). The “P” refers to the phosphodiester link between consecutive nucleotides, although some CpG ODNs have modifications with thiophosphate ester main chains. CpG motifs are unmethylated and considered pathogen-associated molecular patterns (PAMPs). Upon entering the bloodstream, the CpG motifs bind to Toll-like receptor 9 (TLR9), triggering the immune response and causing inflammation. CpG-DNA in the intestine is acidic and negatively charged, constructing the basis of the disclosure.

The term “lipopolysaccharides (LPS)” as used herein, refers to large molecules rich in a plurality of phosphate groups, lipid motifs on oligosaccharides, which carry negative charges. Following damage to the intestinal barrier, increased intestinal permeability allows endotoxins to enter the bloodstream, activating TLR4 receptors, thereby causing systemic inflammation, local tissue inflammation, tissue necrosis, and even organ failure. Studies have also found that high levels of endotoxin in bloodstream is related to insulin resistance for type-2 diabetes. Long-term insulin resistance and high blood sugar levels are key factors developing hepatic steatosis or fatty liver. Obesity is often associated with sustained and systemic inflammation, in part due to disrupted gut microbiota and entry of endotoxins into the bloodstream.

PAMPs, including endotoxins, can be sequestrated by the disclosed API in formulation, through elimination via fecal excretion, so that the entry of PAMPs into the bloodstream is reduced. The formulation is therefore applied for alleviation or treatment of diseases caused by the bacterial toxins in the intestine, including liver injury and failure, fibrosis and cirrhosis, and metabolic liver diseases such as alcoholic liver diseases and non-alcoholic liver diseases, and other severe conditions. The formulation can be also used to treat metabolic diseases caused by intestinal toxin entry into the bloodstream, which include diabetes, obesity, inflammatory bowel diseases and others.

The API in formulation can (1) alleviate liver injury caused by different PAMPs, including endotoxin-induced liver injury, such as acute and chronic liver failure, virus-induced liver failure, drug-induced liver injury, alcohol-induced liver failure, and alcoholic hepatitis; (2) inhibit sepsis caused by PAMPs, including burn-induced sepsis and multi-organ failure; (3) suppress cancer growth and metastasis caused by intestinal PAMP; (4) ameliorate liver fibrosis and cirrhosis, pancreatic fibrosis, and other organ fibrogenesis; (5) reduce endotoxin levels in the bloodstream, of the patients with various diseases, thereby leading to reduced endotoxemia, lowered transaminase levels in bloodstream, and improvement of metabolic functions; (6) alleviate hepatic steatosis; (7) attenuate systemic inflammation, restoring insulin sensitivity, as a novel treatment for diabetes and obesity; and (8) restore the gut microbiota in eubiosis, as a basis for alleviating various diseases. The formulation is administered orally. The above treatment involves administering a specific dose of the API in its formulation to improve the symptoms or treat various related diseases.

The cationic polymer, with its nature of hydrophobicity and positive charges, can be used for sequestration of various PAMPs in the intestine track, thereby excreting PAMPs into feces. Research indicates that various PAMPs produced by gut microbiota, including endotoxins, are significant contributors to systemic inflammation. Chronic systemic inflammation promotes various diseases, including liver failure, diabetes, fatty liver, tumors, and cancers. Therefore, the API in its formulation through sequestration of intestinal PAMP can be used as therapeutic agent to alleviate systemic inflammation caused by endotoxins and local inflammation from various severe conditions, which consequently promotes tissue repair and regeneration. Due to the high-molecular-weight and non-degradable characteristics in the intestine, the cationic polymer is non-absorbable by the body, and can be excreted in feces, demonstrating its high safety and tolerance, which further constitutes the basis of the pharmaceutic application.

The formulation comprises an active pharmaceutical ingredient (API) and pharmaceutically acceptable excipients. The active pharmaceutical ingredient comprises an effective dose of the cationic polymer, or cationic peptides such as α-defensins 5/6 (DEFA5/6), or the derivatives with amino acid substitutions. The formulation is administered orally.

The cationic polymer is a polymer derived from the polymerization of amine monomers and a cross-linking agent, resulting in repeating units in the form of co-polymers. Preferably, the cationic polymer includes secondary amine, tertiary amine, or quaternary amine. More preferably, the cationic polymer is a polystyrene-based quaternary ammonium salt, such as cholestyramine (CAS No. 11041-12-6), modified polyallylamine, copolymer of ethylenediamine, polyvinylamine, polyallylamine, and polylysine. Alternatively, the active pharmaceutical ingredient comprises colestipol (CAS No. 37296-80-3) or colesevelam (CAS No. 182815-44-7). The active pharmaceutical ingredients in the previous application are used to lower the intestinal bile acids, and thus are used to lower cholesterol levels. The active pharmaceutical ingredient remains intact in the intestine without absorption or degradation. Of importance in this disclosure is that various PAMPs were identified as the new targets for the cationic polymers, which constitutes the basis for the disclosure.

The high molecular polymer has a molecular weight of (1-10)×10⁶ Da in the particle size from 40 to 400 mesh. The cationic polymer has high molecular weight and water insolubility, which are critical structural features for the disclosure, ensuring the safety and tolerance of the formulation. The cationic polymer can pass through the digestive tract without being absorbed, and is fully expelled in feces, which consequently can be used to eliminate the intestinal endotoxins, bile acids, and other acidic components such as short-chain fatty acids.

The cationic polymer has the following characteristics:

1. The cationic polymer carries multiple positive charges conferred by amino groups in the copolymers.

2. The cationic polymer has a backbone consisting of carbon-to-carbon linked by covalent bonds.

3. The cationic polymer is not enzymatically digestible in the digestive tract, allowing for excretion through the digestive tract and preventing absorption by the human body.

4. As an example, the cationic peptide comprises a sequence of an antimicrobial peptide, such as α-defensin-5 (DEFA5) or α-defensin-6 (DEFA6) derived from Paneth cells:

α-defensin-5 (DEFA5): ATCYC RHGRC ATRES LSGVC EISGR LYRLC CR, with three pairs of cysteines forming disulfide bonds between residues at positions 3 and 31, 5 and 20, and 10 and 30; and

α-defensin-6 (DEFA6): AFTCH CRRSC YSTEY SYGTC TMVGI NHRFC CL, with three pairs of cysteines forming disulfide bonds between residues at positions 4 and 31, 6 and 20, and 10 and 30.

The activity of the cationic peptide depends on the arginine (R) residues, hydrophobic amino acid residues, and disulfide bonds. The disclosure further provides modifications of the structures of the defensin, including substitutions of various amino acids, to enhance binding capabilities against various bacterial toxins.

The cationic polymer is capable of sequestrating virulence factors produced by the gut microbiota. The virulence factors include, but are not limited to: endotoxin (i.e., lipopolysaccharide), bacterial DNA fragments (containing unmethylated CpG motifs), acidic flagellin proteins, viral DNA, and viral RNA. Preferably, the cationic polymer is administered orally to sequestrate the endotoxins produced by the gut microbiota. Conversely, animal experiments have shown that an uncharged polymer is ineffective, highlighting the importance of the positive charge in the structure of the formulation or API in its therapeutic application. Additionally, the hydrophobic structure of the formulation is also a crucial aspect of the disclosure. The cationic polymer can be selected as a polystyrene-based quaternary ammonium salt, cholestyramine, poly(phenylamine), colestipol, colesevelam, or a cationic peptide such as DEFA5/6, which are used as API to alleviate or treat a variety of diseases caused by PAMP including endotoxin entering the bloodstream and thus for ameliorating or treatment of related diseases.

### EXAMPLE 1

The cationic polymer such as cholestyramine was synthesized as follows:

1. A base copolymer was prepared from styrene and divinylbenzene, with the divinylbenzene acting as a cross-linking agent.

2. The base polymer reacted with a quaternization agent (usually a halide such as methyl chloride) to produce the polystyrene-based quaternary ammonium salt ((4-(3-(4-ethylphenyl)butyl)phenyl)-trimethylazanium), introducing a positively charged quaternary ammonium group.

3. The polystyrene-based quaternary ammonium salt was washed to remove unreacted materials or byproducts. The cationic polymer comprises derivatives of the polystyrene-based quaternary ammonium salt, such as modified polyallylamine and copolymers of diethylenetriamine, with a molecular mass exceeding (1-10)×10⁶ g/mol in the size from 40 to 400 mesh, in the following structure formula:

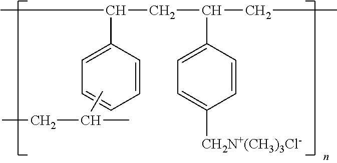

where, the methyl groups on the nitrogen atom can be replaced by a longer hydrocarbon chain. The cationic polymer was administered orally. A dosage for adults is in the range of 1 to 6 grams per dose, 1 to 3 times daily, with a maximum of 18 grams per day. Specific dosages and treatment duration should follow the pharmacopeia guidelines or healthcare provider in prescriptions, adjusted for the severity of the condition and regulatory guidelines.

### EXAMPLE 2

The cationic polymer such as colestipol was synthesized as follows:

1. Preparation of two monomers: diethylenetriamine and epichlorohydrin.

2. Polymerization: the two monomers were polymerized to form a high-molecular-weight copolymer, thereby linking the two monomers together via covalent bonds to form polymer chains.

3. Cross-linking: the polymer chains were connected to each other through chemical bonds, thereby forming a polymer comprising amine groups.

4. Quaternization: the amine groups of the polymer were protonated or alkylated to form a quaternary ammonium compound.

5. Formation of colestipol hydrochloride: the quaternary ammonium compound was treated with hydrochloric acid to form colestipol hydrochloride.

6. Purification: colestipol hydrochloride was purified to remove any unreacted monomers, byproducts, or catalysts; and the purification process involves filtration, washing, and drying.

7. Quality assessment: colestipol hydrochloride was characterized to confirm its properties, molecular weight, and purity; and the characterization process involves techniques such as nuclear magnetic resonance (NMR) spectroscopy, mass spectrometry, and elemental analysis.

The molecular mass of colestipol hydrochloride exceeds 10×10⁶ g/mol, with the molecular formula as follows:

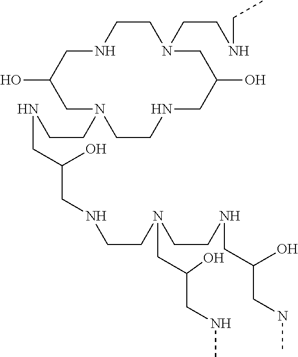

The cationic polymer is utilized to treat cirrhosis, fatty liver diseases, as well as liver fibrosis resulting from non-alcoholic fatty liver.

The cationic polymer is administered orally alone or mixed with food for ingestion during meals. A dosage for adults is in the range of 1 to 8 grams per dose, taken 1 to 3 times per day, with a maximum daily dose of 18 grams. Specific dosages and treatment duration should follow the pharmacopeia guidelines or healthcare provider in prescriptions, adjusted for the severity of the condition and regulatory guidelines.

### EXAMPLE 3

The cationic polymer such as colesevelam was synthesized in following steps. First, poly(allylamine hydrochloride) was dissolved in water, followed by adding sodium hydroxide. Epichlorohydrin was then added to cross-link the polymer, resulting in a clear gel. Second, trimethylamine gas was added to a mixture of tetrahydrofuran and 1,6-dibromohexane. The mixture was heated and stirred, leading to the precipitation of the product. After cooling and filtration, the product was obtained by vacuum drying. Third, the solid product was filtered off and resuspended in methanol. The mixture was then stirred and filtered again. The solid was washed with various solutions including 2M NaCl and deionized water. The polymer was dried to yield the final product.

In the example, colesevelam was used to sequestrate PAMPs, including endotoxins and indole-3-acetic acid produced by the intestinal microbes. Further and through an in vivo experiment, the example confirmed that colesevelam reduced endotoxin levels in the bloodstream, thereby decreasing systemic inflammation, alleviating liver failure and various severe conditions. For example, colesevelam promoted the regression or resolution of NASH and cirrhosis. A dosage for adults is in the range of 1 to 4 grams, administered as granules or tablets, with specific dosage depending on the severity of the condition. The molecular mass of colesevelam exceeds 10×10⁶ g/mol, with the molecular formula as follows

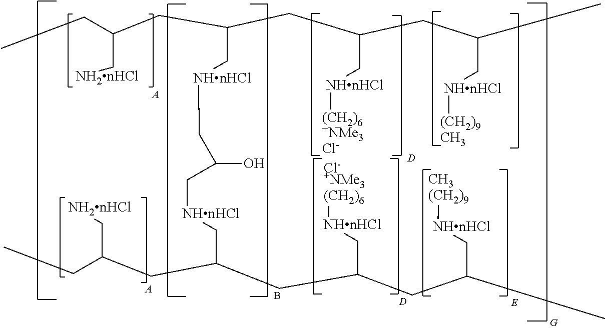

where, A is the primary amine, B is the cross-linked amine, D is the quaternary ammonium alkylated amines, E is the decyalkylated amines, n is the fraction of protonated amines, and G is the extended polymeric network.

### EXAMPLE 4

The formulation of the cationic peptide comprises an antimicrobial peptide, such as α-defensin-5 (DEFA5) or α-defensin-6 (DEFA6) secreted by Paneth cells.

The sequences of α-defensin-5 (DEFA5) and α-defensin-6 (DEFA6) were as follows:

α-defensin-5 (DEFA5): ATCYCRHGRCATRESLSGVCEISGRLYRLCC, with three pairs of cysteines forming disulfide bonds between residues at positions 3 and 31, 5 and 20, and 10 and 30; and

α-defensin-6 (DEFA6): AFTCHCRRSCYSTEYSYGTC TMVGINHRFCCL, with three pairs of cysteines forming disulfide bonds between residues at positions 4 and 31, 6 and 20, and 10 and 30.

The activity of the cationic peptide depends on the arginine (R) residues, hydrophobic amino acid residues, and disulfide bonds. Related chemical modifications thereof, including substitutions of various amino acids in the peptide, can enhance binding capabilities against various bacterial toxins.

### EXAMPLE 5

Direct sequestration of endotoxin by the cationic polymers.

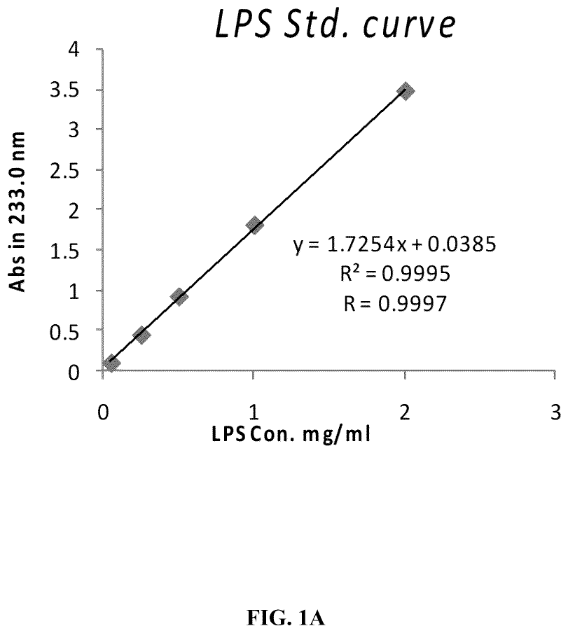

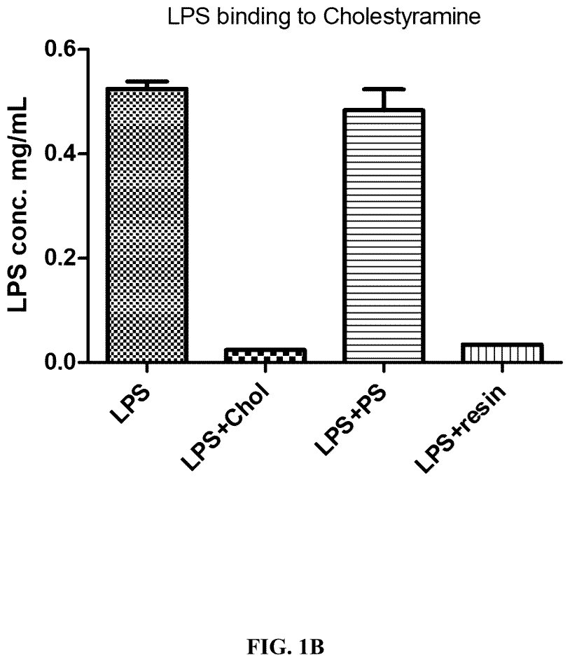

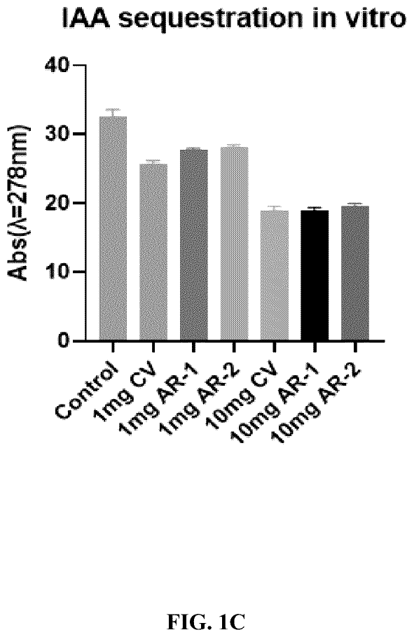

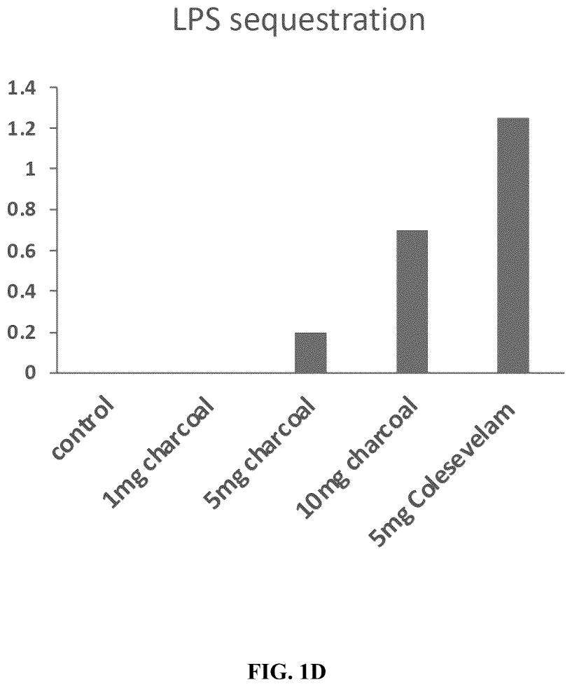

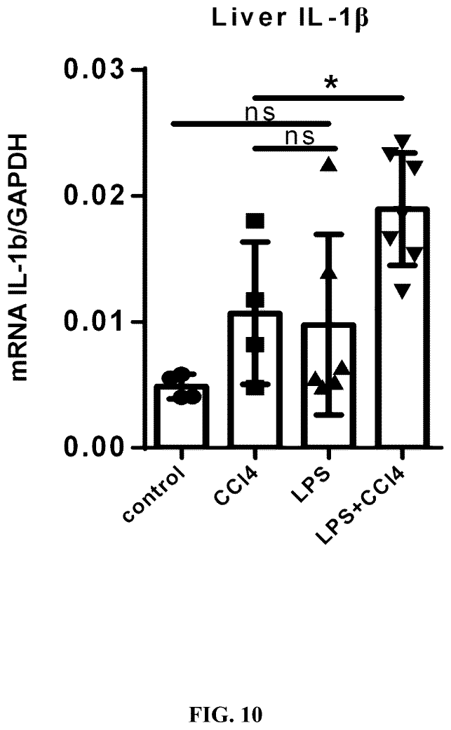

In the example, experiments demonstrated that two types of cationic polymer including cholestyramine (Chol) and colesevelam (CV) sequestered endotoxins (lipopolysaccharides, LPS) as active pharmaceutical ingredient (API). FIG. 1A shows a standard curve for determining the concentration of LPS (endotoxin) in solution. FIG. 1B shows the levels of the remaining LPS in the supernatant after incubating colesevelam (Chol) with endotoxin and separating by centrifugation. As shown in FIG. 1A-1D, 50 mg of cholestyramine (Chol), polystyrene (PS), or polymyxin-B crosslinked beads (Catalog no. 88270, Thermo Fisher Scientific) were incubated respectively with 0.5 mg/mL of lipopolysaccharides (B4, LPS, catalog no. L3012; Sigma-Aldrich) derived from E. Coli. After centrifugation, the levels of the remaining LPS in the supernatant were measured by Limulus Amebocyte Lysate kit. FIG. 1C shows the ability of colesevelam (CV) to sequestrate indole-3-acetic acid, a secondary metabolite produced by the intestinal microbes. Another in vitro experiment of the example showed that administration of colesevelam depleted certain secondary metabolites derived from the intestinal microbes. FIG. 1D shows the ability of colesevelam (CV) and activated charcoal used to sequestrate endotoxins (LPS) in vitro. Furthermore, and referring to FIG. 10, in additional experiment, 10 mg of colesevelam adsorbed about 2 mg of endotoxins, demonstrating that colesevelam and cholestyramine in a common mechanism of action used for sequestration of intestinal PAMPs and secondary metabolites from the intestinal microbes.

### EXAMPLE 6

Oral administration of cholestyramine reduced endotoxins levels in bloodstream at the condition of metabolic syndrome.

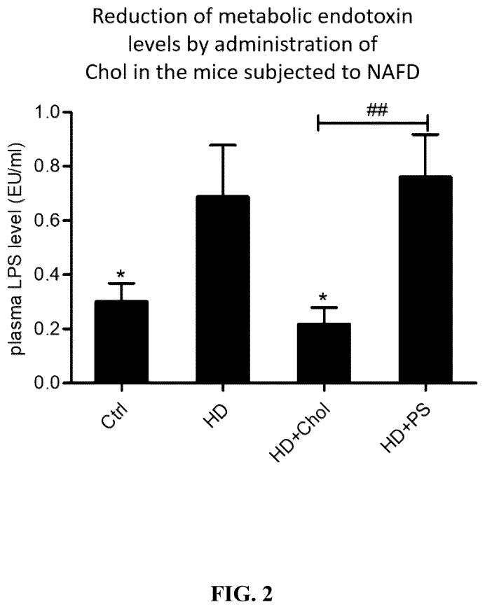

Research found that vitamin D deficiency can impair the innate immune functions in mice, featured as reduced expression of antimicrobial peptides by Paneth cells in the small intestine, which consequently lead to elevated endotoxin levels in the bloodstream for systemic inflammation, and various metabolic diseases. In one experiment, male BALB/c mice were fed a vitamin D-deficient and high-fat diet for 10 weeks. Then, some mice were supplemented with cholestyramine at 3% w/w in the chow for an additional 8 weeks. At the end of experiment, the endotoxin levels were measured using the Limulus Amebocyte Lysate (LAL) test. The mice subjected to vitamin D deficiency and a high-fat diet feeding exhibited typical metabolic syndrome symptoms, including insulin resistance, central obesity, and fatty liver. FIG. 2 illustrate that oral administration of cholestyramine can reduce the systemic levels of endotoxin, presumably through sequestration the intestinal endotoxin.

### EXAMPLE 7

Oral administration of cholestyramine maintained the gut microbiota in balance.

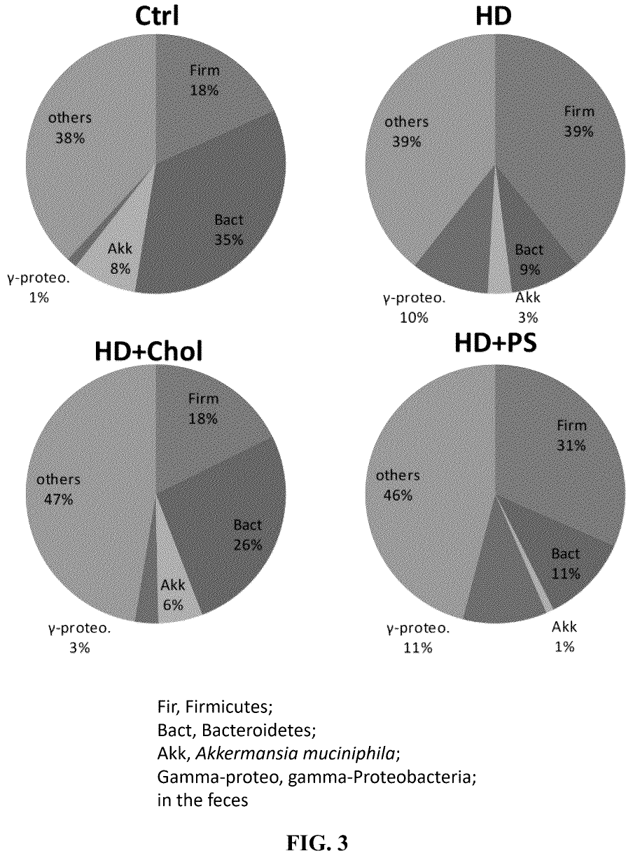

In one experiment, male BALB/c mice were fed a vitamin D-deficient-high-fat diet for 10 weeks to generate metabolic syndrome. Then, some mice were supplemented with cholestyramine at 3% w/w in the high fat chow for an additional 8 weeks. Subsequently, the mice subjected to the feeding exhibited dysbiosis of gut microbiota, showing reduced abundance of symbiotic bacteria (Akkermansia muciniphila, AKK) and increased abundance of Firmicutes. FIG. 3 illustrates the result of 16S-rDNA analysis of fecal microbiota from the mice. Research found that under condition of the high-fat feeding, Paneth cells in the ileum produced fewer antimicrobial peptides, causing dysbiosis of gut microbiota and bacterial overgrowth. Consequently, PAMPs released by intestinal microbes in the small intestine was absorbed into the liver through portal vein. However, administration of cholestyramine helps to restore the eubiosis of gut microbiota, as indicated by increased levels of Akkermansia muciniphila and reduction the abundance of Firmicutes and gamma proteobacteria among others.

### EXAMPLE 8

Oral administration of cholestyramine alleviated non-alcoholic hepatic steatosis.

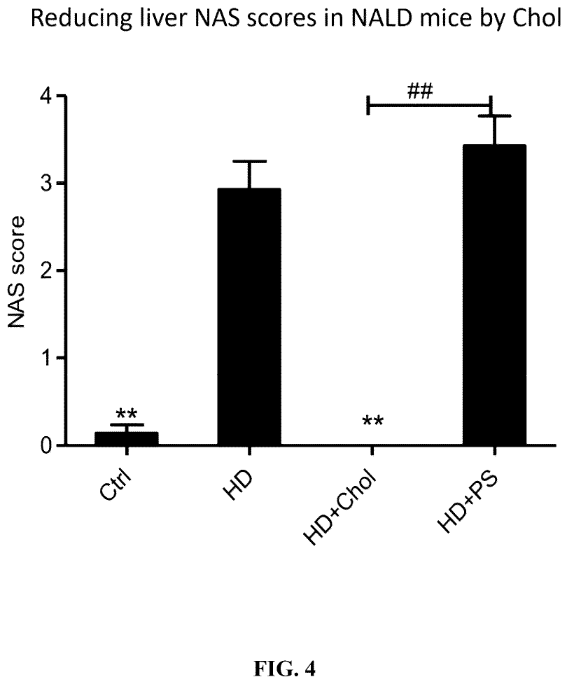

In one experiment, male BALB/c mice were fed a vitamin D-deficient-high-fat diet for 10 weeks. Then, some mice were supplemented with cholestyramine at 3% w/w in the high fat chow for an additional 8 weeks. The mice subjected to vitamin D-deficient-high-fat diet exhibited typical hepatic steatosis (fatty liver). However, as shown in FIG. 4, administration of the cholestyramine (Chol) alleviated fatty liver as indicated by NAS scores. Various factors contribute to pathogenesis of fatty liver, including endotoxin-induced insulin resistance and hypertriglyceridemia, which consequently promotes hepatic steatosis. The experimental results showed that cholestyramine, presumably through sequestration of intestinal PAMPs relieved the high-fat-diet induced fatty liver as indicated by NAS scores.

### EXAMPLE 9

Oral administration of cholestyramine alleviated hepatic inflammation exerted by metabolic syndrome.

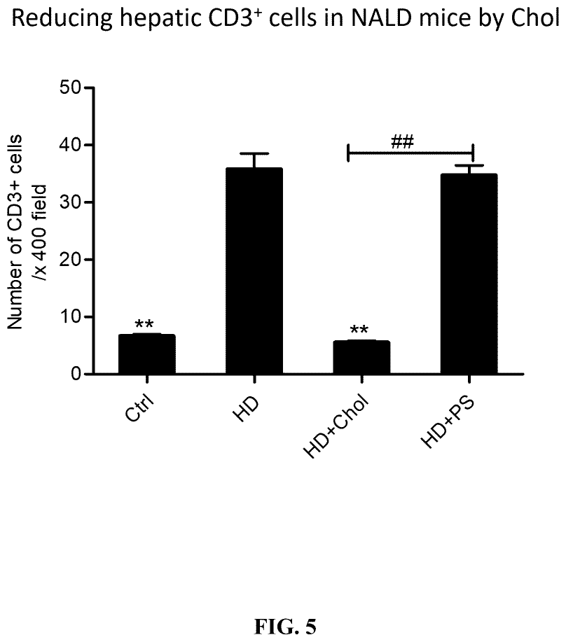

Normally, in the healthy liver there are few T cells (CD3+ cells). After liver injury, large amount of T cells can migrate into the liver, guided by chemokines released in the liver tissue by innate immune cells, to initiate immune surveillance. In one experiment, male BALB/c mice were fed a vitamin D-deficient and high-fat diet for 10 weeks. And then, some mice were supplemented with cholestyramine at 3% w/w in the high fat chow for an additional 8 weeks. The mice subjected to the high-fat feeding showed persistent hepatic inflammation, indicated as increased number of CD3+ T cells in the liver. However, oral administration of cationic polymer reduced the intrahepatic infiltration of T cells. FIG. 5 shows the distribution of CD3+ cells in the liver tissues.

### EXAMPLE 10

Oral administration of cholestyramine suppressed the elevated level of TNF-α in metabolic syndrome.

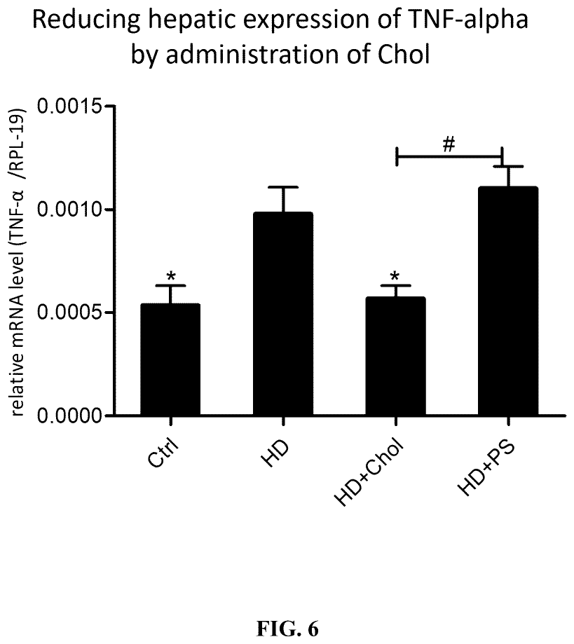

In one experiment, adult BALB/c mice were fed a vitamin D-deficient-high-fat diet for 10 weeks. And then some mice were supplemented with the cholestyramine at 3% w/w in the high fat chow for an additional 8 weeks. The mice subjected to the high-fat diet showed persistent hepatic inflammation, determined as increased expression of inflammatory cytokine, TNF-α in liver. However, as shown in FIG. 6, the cationic polymer lowered the mRNA levels of TNF-α in the liver. The experimental results can be explained by a chain of events from sequestration of intestinal endotoxins to amelioration of Toll-like receptor/CD14 signaling pathway, leading to reducing the expression of the inflammatory cytokine TNF-α.

### EXAMPLE 11

Oral administration of cholestyramine alleviated liver injury and reduce hepatic transaminase levels in the condition of metabolic syndrome and non-alcoholic steatohepatitis.

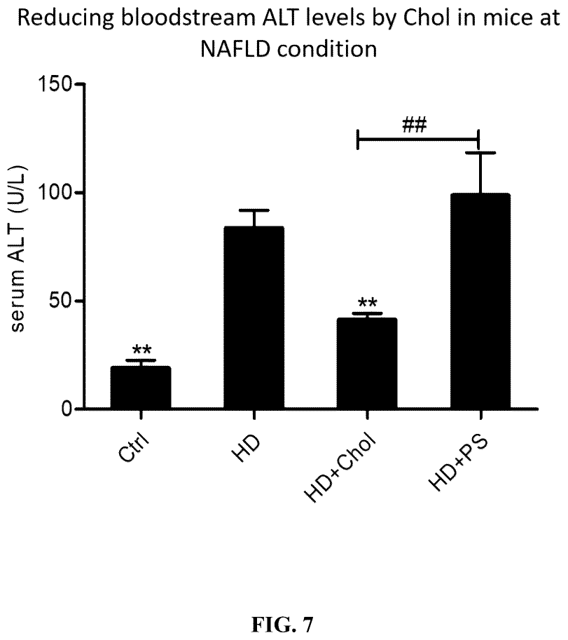

Elevated transaminase level is a common biochemical marker for various hepatic injuries. As shown in FIG. 7, long-term feeding with vitamin D deficient and high-fat diet led to liver injury, indicated as increased level of bloodstream transaminase. However, oral administration of cationic polymer mitigated liver cell damage and reduced the level of alanine aminotransferase (ALT). Increased level of metabolic endotoxin in the bloodstream is a common mechanism for various forms of hepatitis, including viral hepatitis, drug-induced hepatitis, autoimmune hepatitis, alcoholic hepatitis, non-alcoholic steatohepatitis, liver injury resulting from failure, cirrhosis, and fatty liver. Thus, the disclosed method herein can be used to alleviate the mentioned hepatitis as well.

### EXAMPLE 12

Endotoxin (LPS) can exacerbate the drug-induced cirrhosis as determined by fibrotic histological analysis.

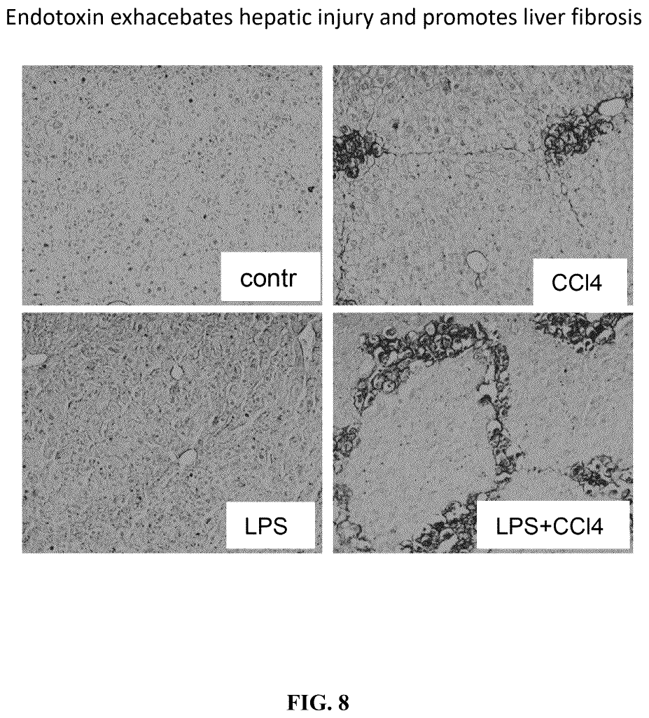

In another experiment, adult male BALB/c mice were injected intraperitoneally with escalated dosage of carbon tetrachloride (CC14 from 1.0 to 2.5 ml/kg body weight) for 2 weeks. And then some mice were given lipopolysaccharides injection (LPS at 0.3 mg/kg, i.p.) for an additional 2 weeks. Under sustained injury conditions, some mice were supplemented with cholestyramine at 3% w/w in the chow for another 4 weeks. FIG. 8 shows Sirius Red staining of liver tissues. It is well known that hepatic injury induced activation of hepatic stellate cells (HSCs) mediate hepatic fibrosis. As shown, oral administration of cationic polymer alleviated the drug-induced liver injury and liver fibrosis, presumably through intestinal sequestration of PAMP, which are the critical factors in promoting liver fibrosis and activation of hepatic stellate cells.

### EXAMPLE 13

Endotoxin (LPS) exacerbates the drug-induced cirrhosis as measured by expression of type-I collagen at mRNA levels.

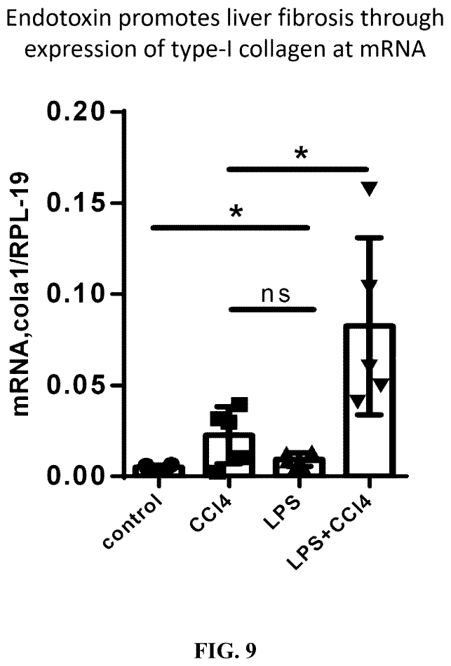

In one experiment, adult male BALB/c mice were intraperitoneally injected with escalated dosage of carbon tetrachloride (CC14, 1.0-2.5 ml/kg body weight) for 2 weeks. Then, some adult male BALB/c mice were given additional lipopolysaccharides (LPS, 0.3 mg/kg, i.p.) for 2 weeks. At such condition, some mice were supplemented with cholestyramine at 3%w/w in the chow for 4 weeks. FIG. 9 shows the expression levels of collagen gene (type-1 collagen, α-1) in liver tissue. In normal liver tissues, HSCs are quiescent, featured as storing vitamin A and producing type-4 collagen and other loose extracellular matrix (ECM). The high expression of collagen and the formation of fibrotic septa are common in various liver diseases. And the results showed that cationic polymer alleviated the endotoxin-mediated liver fibrogenesis. Endotoxemia is common in various hepatic fibrosis and cirrhosis, caused by alcohol, drugs, viruses (such as HBV and HCV), and immune system disorders. Therefore, the current disclosure can be applied for treatment of various forms of liver fibrosis.

### EXAMPLE 14

Endotoxin (LPS) exacerbates the drug-induced hepatic inflammation, measured as the mRNA expression of IL-1B in the liver.

In one experiment, adult male BALB/c mice were intraperitoneally injected with escalated levels of carbon tetrachloride (CC14, 1.0-2.5 ml/kg body weight) for 2 weeks. Then, some mice were given lipopolysaccharides (LPS, 0.3 mg/kg, i.p.) for an additional 2 weeks. Under the condition, some mice were supplemented with cholestyramine at 3 w/w. % in the chow for an additional 4 weeks. As shown in FIG. 10, endotoxins enhance the mRNA expression of interleukin-1β (IL-1β) caused by drug-induced liver injury. It is known that PAMPs activate many types of cells to produce interleukin-1 (IL-1), leading to fever, cytokine storms, and tissue damage. In this current disclosure, the cationic polymer in the oral administration can be used for sequestration of intestinal endotoxins, which consequently can alleviate drug-induced liver inflammation, a common mechanism of action for many types of liver injury and fibrogenesis.

### EXAMPLE 15

Endotoxin weakens the innate immune system in the intestine and suppresses expression of alpha-defensin 5 by Paneth cells.

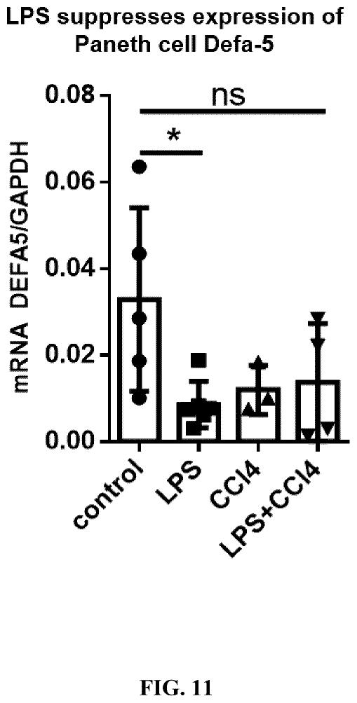

In one experiment, adult male BALB/c mice were intraperitoneally injected with escalated dosage of carbon tetrachloride (CC14 from 1.0 to 2.5 ml/kg body weight) for 2 weeks. Then, some mice were given lipopolysaccharides (LPS, 0.3 mg/kg, i.p.) for an additional 2 weeks. As shown in FIG. 11, liver injury and LPS respectively suppressed the expression levels of α-defensin-5 (DEFA5) produced by Paneth cells in the ileum tissue. Lower DEFA5 levels worsen gut microbiota into dysbiosis state, and conversely, increased level of endotoxin may contribute to liver inflammation, leading to tissue injury and fibrogenesis.

### EXAMPLE 16

Endotoxin suppressed the expression of mucin gene by Goblet cells in the intestine.

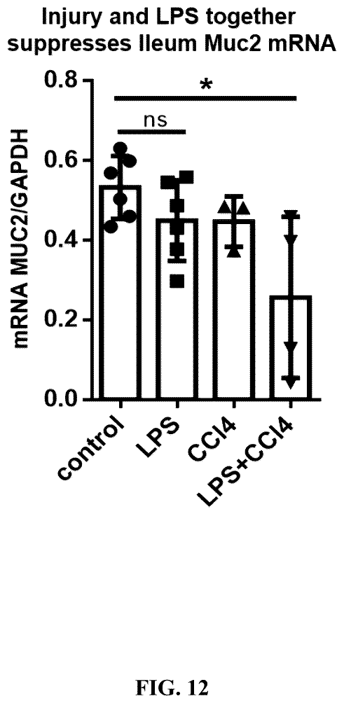

Clinical research has found that the innate immune system in the intestine by the elderly is compromised along with aging, which may damage the intestinal epithelial cells, resulting in influx of endotoxin into portal vein. In one experiment, adult male BALB/c mice were intraperitoneally injected with escalated dosage of carbon tetrachloride (CC14 from 1.0 to 2.5 ml/kg body weight) for an additional 2 weeks. Then, some mice were given lipopolysaccharides (LPS, 0.3 mg/kg, i.p.) for an additional 2 weeks. As shown in FIG. 12, the drug-induced liver injury and endotoxin respectively reduced the mRNA expression of Muc-2 by Goblet cells in the ileum tissue, according to RT-qPCR analysis.

### EXAMPLE 17

Endotoxin suppressed the expression of tight junction proteins in intestinal epithelial cells.

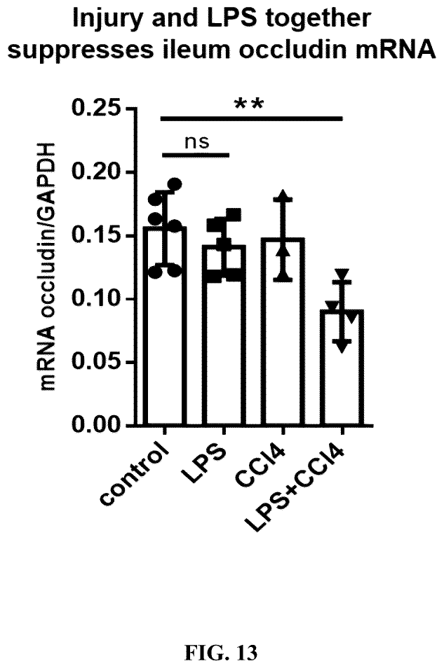

The tight junction proteins are essential components of intestinal epithelial cells and the innate immune system. In one experiment, adult male BALB/c mice were intraperitoneally injected with carbon tetrachloride (CC14, 1.0-2.5 ml/kg body weight) for 2 weeks. Then, some mice were given lipopolysaccharides (LPS, 0.3 mg/kg, i.p.) for an additional 2 weeks. As shown in FIG. 13, the drug-induced liver injury and endotoxins suppressed the mRNA expression of occludin gene for the tight junction in the ileum tissues, according to RT-qPCR analysis.

### EXAMPLE 18

Oral administration of cholestyramine ameliorated the hepatic-toxin exerted hepatic fibrogenesis, measured as histology.

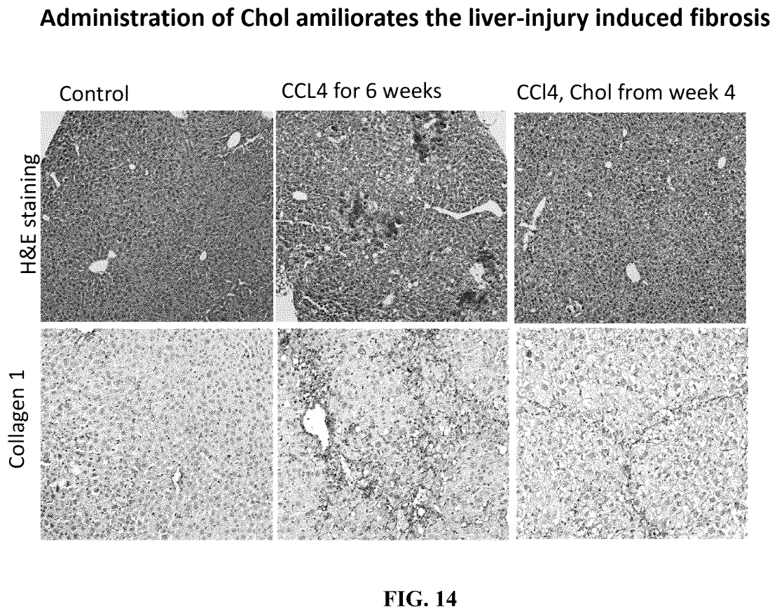

Endotoxemia is common in various liver diseases, including viral hepatitis, alcoholic hepatitis, or drug-induced hepatitis. Our previous study has also found that the pro-inflammatory such as cytokine IL-1 can trigger TGF-β activation through MMP13 expression the latent complex, which consequently promotes trans-differentiation of hepatic stellate cells for fibrosis. In another experiment, adult male BALB/c mice were intraperitoneally injected with escalated dosage of carbon tetrachloride (CC14 from 1.0 to 2.5 ml/kg body weight) for 6 weeks. Then, some mice were supplemented with cholestyramine at 3%, w/w in the chow for an additional 4 weeks. FIG. 14 shows Sirius Red staining and type-I collagen staining of the liver tissues. As shown in FIG. 14, oral administration of cationic polymer as cholestyramine suppressed trans-differentiation of HSC activation, and alleviate cirrhosis, presumably through intestinal sequestration of intestinal PAMP.

### EXAMPLE 19

Oral administration of cholestyramine suppressed the hepatic-toxin-exerted expression of hepatic type-1 collagen.

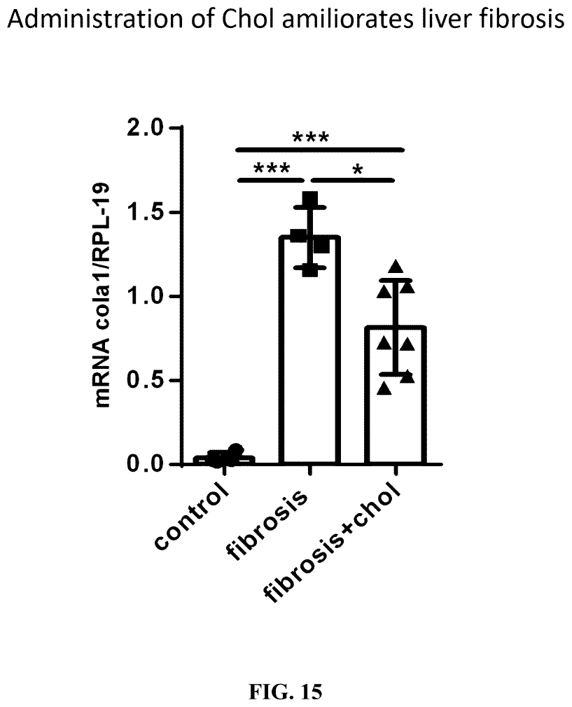

Studies have found that the differentiated HSCs increase the expression of collagen genes, leading to the formation of fibrotic septa. In one experiment, adult male BALB/c mice were given intraperitoneal injections of carbon tetrachloride (CC14) for 6 weeks. In the condition of continued exposure to CC14, some mice were supplemented with cholestyramine at 3%, w/w in the chow for an additional 4 weeks. FIG. 15 shows the expression levels of collagen gene (type-1 collagen, α-1) in the liver tissues were suppressed by oral administration of cationic polymer as API.

### EXAMPLE 20

Oral administration of cholestyramine reduced the levels of alanine aminotransferase (ALT) in the condition of drug-mediated liver injury.

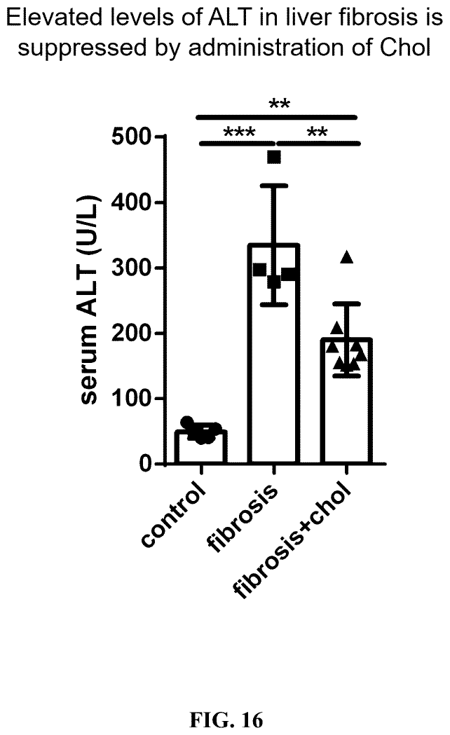

The elevation of ALT in the bloodstream is a clinical indicator of liver injury, including drug-induced hepatitis, viral hepatitis, and autoimmune hepatitis. In one experiment, adult male BALB/c mice were given intraperitoneal injections of carbon tetrachloride (CC14) for 6 weeks. Then, some mice were fed a diet containing the cholestyramine at 3%, w/w in the chow for an additional 4 weeks. As shown in FIG. 16, the cationic polymer binds to the intestinal endotoxins, mitigating liver cell damage and reducing ALT levels. Consequently, the cationic polymer is utilized to reduce the levels of ALT resulting from liver injury.

### EXAMPLE 21

Oral administration of cholestyramine reduced the levels of aspartate aminotransferase (AST) from liver injury.

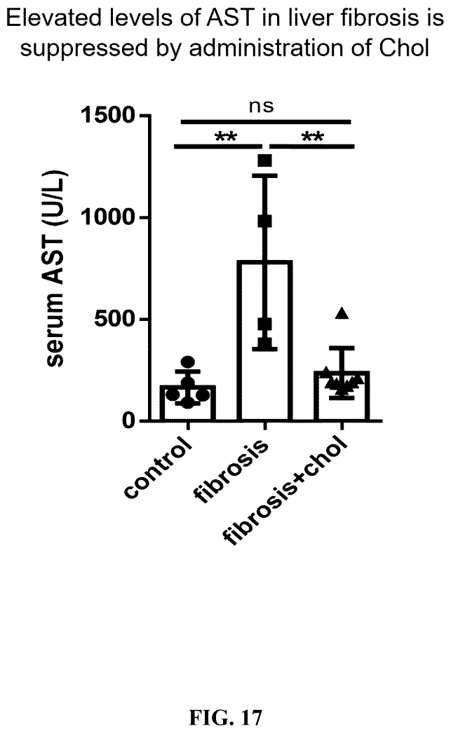

Although the elevation of both ALT and AST is used as indicators for liver injury, ALT is more liver-specific, and its elevation is an early and more reliable sign of liver cell damage. Elevated AST levels occur in a broader range of liver injuries and can even be influenced by damage to other tissues. The AST/ALT ratio elucidates the nature and severity of liver injury. In one experiment, adult male BALB/c mice were given intraperitoneal injections of carbon tetrachloride (CC14) for 6 weeks. Then, some mice were supplemented with cholestyramine at 3%, w/w in the chow for an additional 4 weeks. As shown in FIG. 17, the cationic polymer alleviated drug-induced liver injury and reducing AST levels.

### EXAMPLE 22

Oral administration of cholestyramine reduced total bilirubin (TBIL) levels from liver injury.

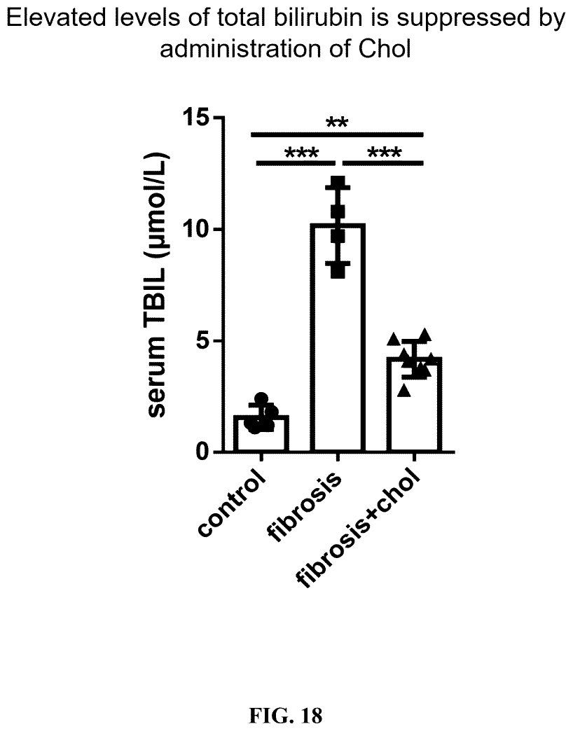

Total bilirubin is an important indicator for assessing liver function, comprising direct bilirubin (DBIL) and indirect bilirubin (IBIL). Elevated total bilirubin levels are often associated with liver injury, reflecting issues in bilirubin metabolism and excretion by the liver. When liver diseases occur, the ability of the liver cells to process and excrete bilirubin decreases, raising the levels of total bilirubin in the blood. The liver diseases include, but not limited to, acute hepatitis, chronic active hepatitis, cirrhosis, liver cancer and drug-induced liver injury. In one experiment, adult male BALB/c mice were given intraperitoneal injections of carbon tetrachloride (CC14) for 6 weeks. Then, some mice were supplemented with cholestyramine at 3 w/w. % in the chow for an additional 4 weeks. As shown in FIG. 18, the cationic polymer mitigated liver injury, showing reduction the levels of total bilirubin in the blood.

### EXAMPLE 23

Oral administration of cholestyramine reduced the levels of direct bilirubin (DBIL) from liver injury.

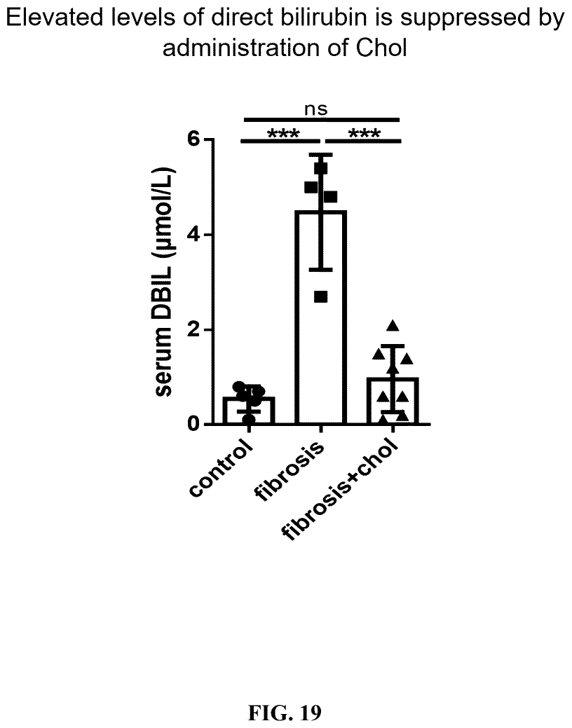

Direct bilirubin is a product of bilirubin metabolism in the liver. Bilirubin mainly comes from the hemoglobin released when aged red blood cells break down. Bilirubin is converted to indirect bilirubin (IBIL) and further conjugated with glucuronic acid to form direct bilirubin. Bile duct obstruction, liver inflammation and cirrhosis can cause elevated levels of direct bilirubin. In one experiment, adult male BALB/c mice were given intraperitoneal injections of carbon tetrachloride (CCl₄) for 6 weeks. Then some mice were supplemented with cholestyramine at 3 w/w. % in the chow for an additional 4 weeks. As shown in FIG. 19, the cationic polymer mitigated liver cell damage and reduced the levels of direct bilirubin in the blood.

### EXAMPLE 24

The spike protein from Sars-Covid-2 virus increased the expression of the pro-inflammatory cytokine IL-1B.

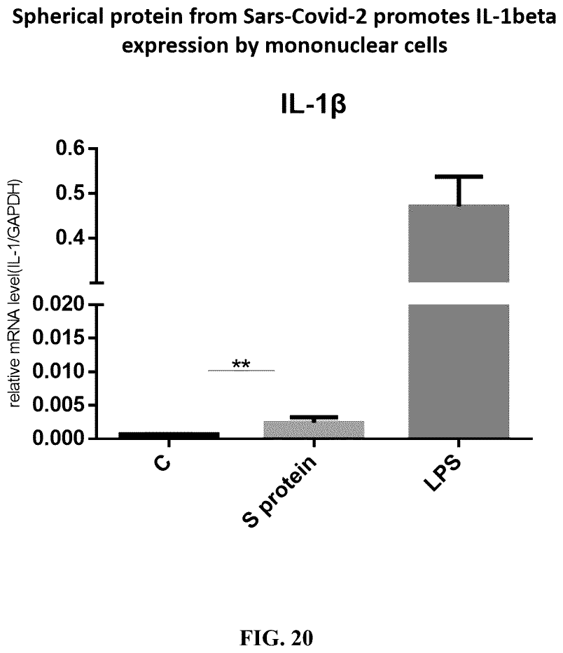

Reports have found that in COVID-19 patients, the cytokine storm is a severe complication involving the excessive production of the pro-inflammatory cytokines (e.g., IL-6, IL-1B, and TNF-α), leading to intestinal injuries, increased intestinal permeability, and endotoxin translocation into the bloodstream, further triggering a systemic cytokine storm. As shown in FIG. 20, the spike (S) protein from Sars-Covid-2 virus activates monocytes, increasing the mRNA expression of the pro-inflammatory cytokine IL-1B, according to RT-qPCR analysis.

### EXAMPLE 25

The spike protein from Sars-Covid-2 virus increased the expression of the pro-inflammatory cytokine TNF-α.

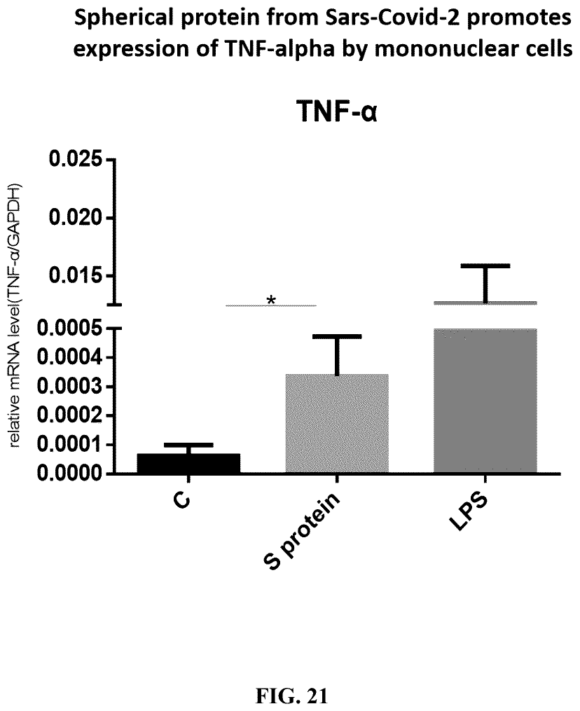

Spike (S) protein from coronavirus is highly immunogenic and can induce a cytokine storm. Coronavirus may synergize with endotoxins to amplify the inflammatory response. A cell experiment was conducted to measure the expression of the pro-inflammatory cytokine TNF-α in monocytes activated by the spike protein and LPS. FIG. 21 shows that the spike protein increased the mRNA expression of TNF-α by more than 5 times, while LPS increases the mRNA expression of TNF-α by about 8 times, according to RT-qPCR analysis. Clinical studies have reported that extracorporeal blood circulation to remove the endotoxins was successfully used to treat the severe COVID-19 patients in ICU. Therefore, the cationic polymer as API can be applied to eliminate the endotoxins, reduce pro-inflammatory cytokines, and treat the symptom of COVID-19 caused by intestinal endotoxin.

### EXAMPLE 26

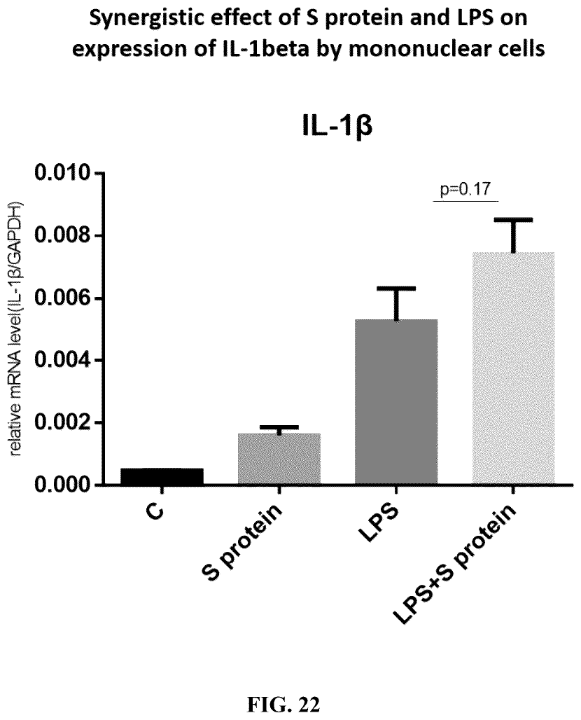

The spike protein and endotoxins synergize to activate monocytes, increasing the expression of the pro-inflammatory cytokine interleukin-1 (IL-1B). Similarly, interleukin-1 (IL-1) is also an important pro-inflammatory cytokine. IL-1B, produced by various immune cells in response to LPS and viral immunogens, induces the expression of multiple MMP genes, leading to ECM degradation, cell apoptosis, and tissue damage. Clinical studies have found that a close link between cytokine storm in severe COVID-19 patients and IL-1 levels. Experiments were conducted to measure the expression of IL-1B in immune cells treated with the spike protein and LPS, both individually and in combination with the spike protein. FIG. 22 shows that the spike protein increased the mRNA expression of IL-1B by about 5 times, while LPS increases mRNA expression of IL-1B by about 9 times. A combination of the spike protein and LPS further increases the mRNA expression of TNF-a by about 16 times. Therefore, the cationic polymer can be applied to remove the intestinal endotoxins, alleviating symptom by severe COVID-19 patients.

### EXAMPLE 27

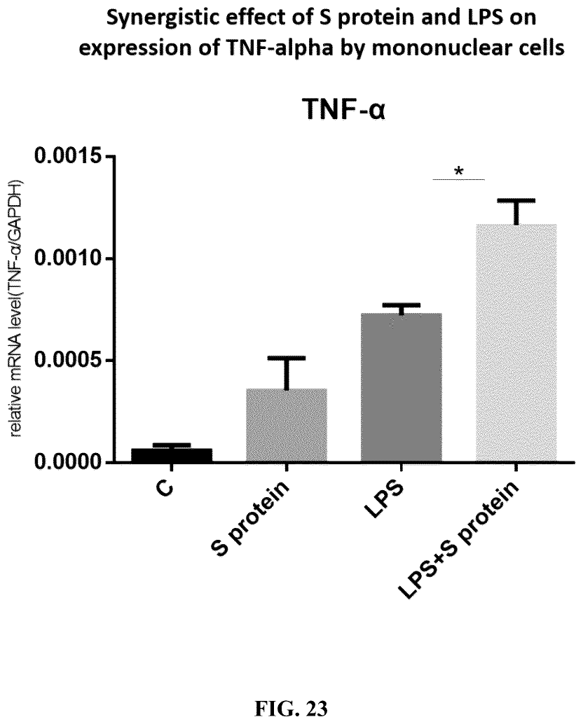

The spike (S) protein and endotoxins synergize to activate monocytes, increasing the expression of the pro-inflammatory cytokine TNF-α. Similar to Example 26, experiments were conducted to measure the synergistic effect of endotoxins and the spike protein on the regulation of the pro-inflammatory cytokine TNF-α. As shown in the FIG. 23, the spike protein increases the mRNA expression of TNF-α by about 6 times, while LPS increases the mRNA expression of TNF-α by about 10 times. A combination of the spike protein and LPS further increases the mRNA expression of TNF-α, promoting the cytokine storm in vivo. Therefore, the cationic polymer as API can be used to remove intestinal endotoxins, alleviating symptom from severe COVID-19 infection.

### EXAMPLE 28

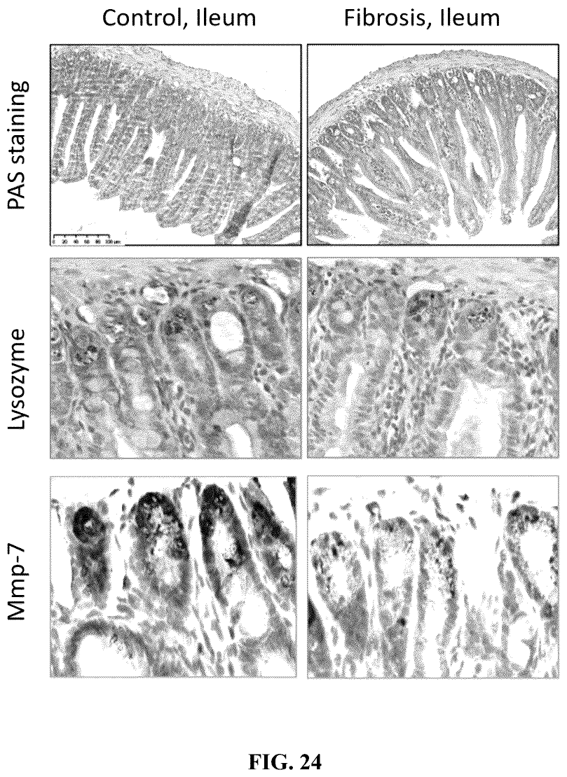

Liver injury reduces the phagocytic function of Paneth cells, impacting the innate immune system in the intestine. Paneth cells, located in the ileum of the small intestine, secrete antimicrobial peptides and Wnt proteins. The antimicrobial peptides, including α-defensin-5 and α-defensin-6, maintain intestinal microbial balance. In liver diseases, the immune system in the intestine may be compromised, leading to impaired intestinal permeability, known as “leaky gut”. The leaky gut allows bacteria and endotoxins to enter the bloodstream, activating the immune system. The immune system comprises Paneth cells in the gut. In one experiment, adult male BALB/c mice were given intraperitoneal injections of carbon tetrachloride (CC14, 1.0-2.5 ml/kg body weight) for 8 weeks. FIG. 24 displays Periodic acid-Schiff (PAS) staining of the mouse ileum tissues to highlight the intestinal mucosa. Experiments also assessed Lysozyme staining to show the phagocytic activity of epithelial cells, while matrix metalloproteinase-7 (Mmp-7) staining to indicate the expression levels of α-defensin secreted by Paneth cells. As shown in FIG. 24, liver cirrhosis reduces the function of Paneth cells in the ileal tissues, impacting the innate immune system.

### EXAMPLE 29

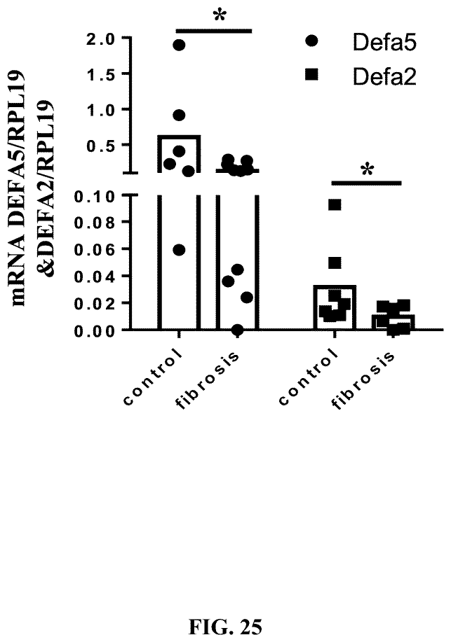

Paneth cells are resided in the crypt in the small intestine. The secretory granules contain the antimicrobial peptides, such as defensins and lysozymes, which have bactericidal functions against gut microorganisms. In one experiment, adult male BALB/c mice were given intraperitoneal injections of carbon tetrachloride (CC14, 1.0-2.5 ml/kg body weight) for 8 weeks. As shown in FIG. 25, liver injury and fibrosis reduced the mRNA expression of α-defensin-2 and α-defensin-5, indicating impaired function of Paneth cells. Therefore, administration of α-defensins, as an active pharmaceutical ingredient (API), can balance the gut microbiota, inhibit the overgrowth of microorganisms in small intestine, and directly bind certain PAMPs in pharmaceutical application.

### EXAMPLE 30

Oral administration of synthetic human α-defensin-5 (DEFA5) to mitigate liver fibrosis.

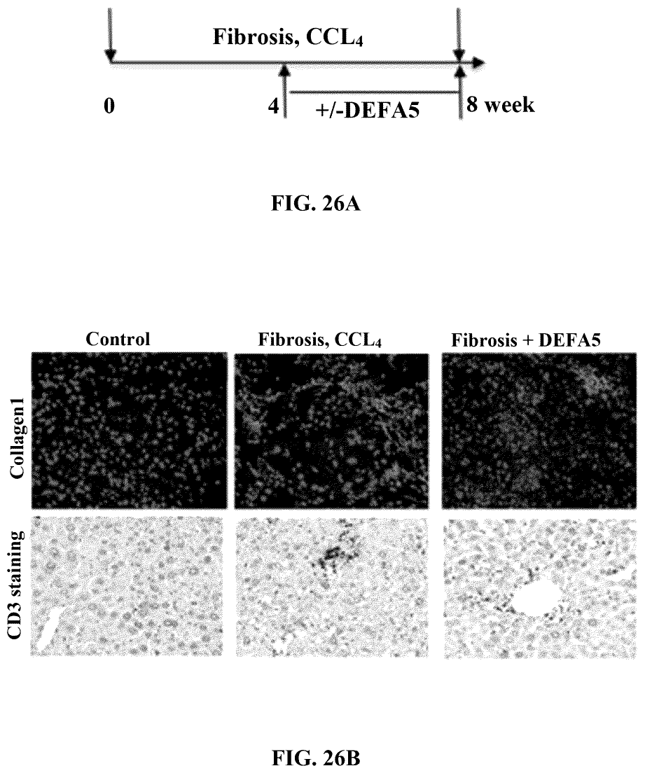

Paneth cells secret innate immune substances, such as antimicrobial peptides, to maintain intestinal homeostasis. The epithelial cells of the small intestine and the innate immune substances maintain the gastrointestinal barrier function, protect against the penetration of commensal and pathogenic bacteria, and safeguard intestinal epithelial cells. The antimicrobial peptides can suppress the small intestinal overgrowth of bacteria (SIBO), a common pathologic condition for many metabolic diseases. In one experiment, adult male BALB/c mice were given intraperitoneal injections of carbon tetrachloride for 4 weeks. While continuing administration of carbon tetrachloride, some mice were orally given synthetic human α-defensin-5 (10 mg, twice a week) for an additional 8 weeks (shown in FIG. 26A). FIG. 26B shows Sirius Red staining for liver fibrosis and CD3+ staining of liver tissue. The experimental results showed that administration of human α-defensin-5 (DEFA5) as API reduced liver fibrosis caused by liver injury. The mechanism of action by the DEFA5 as API can be explained in part by inhibiting PAMPs, thereby reducing liver inflammation and damage, which in turn reduces the transdifferentiation of HSC and lowers liver fibrosis score.

### EXAMPLE 31

Oral administration of synthetic human α-defensin-5 (DEFA5) as API to reduce intestinal permeability.

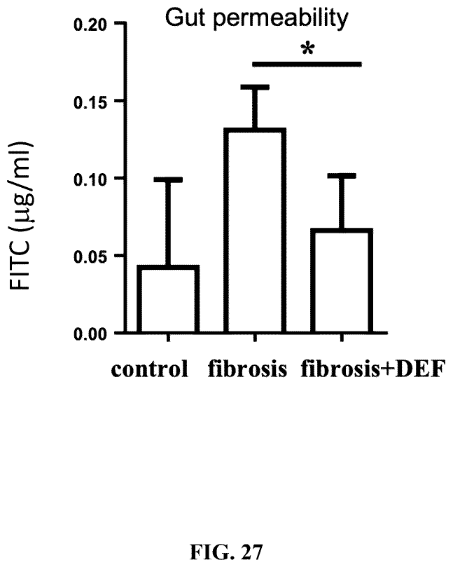

In one experiment, adult male BALB/c mice were given intraperitoneal injections of carbon tetrachloride for 4 weeks; while continuing administration of carbon tetrachloride, some mice were given the synthetic human α-defensin-5 (DEFA5) (10 mg, twice a week) for 8 weeks. Finally, the mice were given fluorescein isothiocyanate (FITC). As shown in FIG. 27, the FITC levels in the bloodstream were measured to indicate the intestinal permeability. Studies indicated that liver injury caused by drugs, including carbon tetrachloride, can impair the 25-hydroxylation of vitamin D, leading to a low vitamin D status. Vitamin D deficiency affects the expression of the defensins secreted by Paneth cells, resulting in small intestinal bacterial overgrowth (SIBO) and the translocation of PAMPs into the bloodstream, thereby causing chronic inflammation. Therefore, the defensins, as a therapeutic API, can treat various gastrointestinal disease and alleviate drug-induced liver fibrosis.

### EXAMPLE 32

Oral administration of synthetic human α-defensin-5 (DEFA5) reduced the entry of intestinal endotoxins into the bloodstream caused by liver cirrhosis.

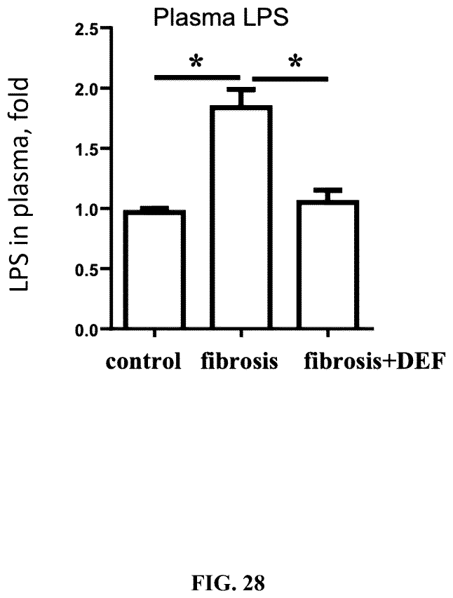

In one experiment, adult male BALB/c mice were given intraperitoneal injections of carbon tetrachloride for 4 weeks. While continuing administration of carbon tetrachloride, some mice were given the synthetic human α-defensin-5 (DEFA5) (10 mg, twice a week) for 8 weeks. As shown in FIG. 28, the endotoxin level in the bloodstream was measured using the Limulus Amebocyte Lysate (LAL) test. The experimental results showed that a low dose of the synthetic human α-defensin-5 (DEFA5) reduced endotoxin translocation into the bloodstream, further alleviating various liver diseases such as liver failure, cirrhosis, and fatty liver.

### EXAMPLE 33

Oral administration of synthetic human α-defensin-5 (DEFA5) was used for balancing gut microbiota.

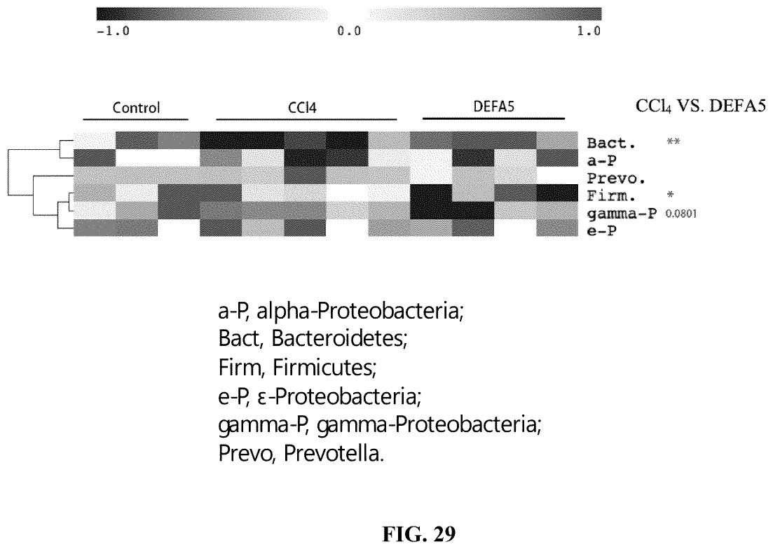

In one experiment, adult male BALB/c mice were given intraperitoneal injections of carbon tetrachloride for 4 weeks; while continuing administration of carbon tetrachloride, some mice were given the synthetic human α-defensin-5 (DEFA5) (10 mg, twice a week) for 8 weeks. As shown in FIG. 29, the distribution of bacterial microbiota in mouse feces was analyzed. The experiment showed that the synthetic human α-defensin-5 (DEFA5) balanced the intestinal bacterial microbiota, increasing the abundance of Bacteroidetes and reducing the abundance of Firmicutes and gamma-Proteobacteria.

It will be obvious to those skilled in the art that changes and modifications may be made, and therefore, the aim in the appended claims is to cover all such changes and modifications.

What is claimed is:

1. A method of treating a subject having a disease or disorder related to or caused by pathogen-associated molecular pattern (PAMP) derived from intestinal microorganisms, the method comprising:
administering to the subject a therapeutically effective amount of a cationic polymer which binds to the PAMP in digestive tract of the subject to form a sequestrant-PAMP complex, so that the sequestrant-PAMP complex is eliminated from the digestive tract.

2. The method of claim 1, wherein the cationic polymer is a cationic copolymer comprising at least an amine group, a cationic peptide, or a derivative thereof.

3. The method of claim 2, wherein the cationic polymer is an organic polymer; and the amine group is in the form of a secondary amine, a tertiary amine, or a quaternary amine.

4. The method of claim 3, wherein the organic polymer is prepared by introducing a nitro group through nitration to an aliphatic or aromatic polymer, and reducing the aliphatic or aromatic polymer comprising the nitro group, to obtain the organic polymer.

5. The method of claim 3, wherein an active pharmaceutical ingredient (API) of an enteric coated formulation of the cationic polymer is resistant to enzymatic degradation and non-absorbable.

6. The method of claim 5, wherein the organic polymer is at least one selected from a group consisting of a polystyrene-based quaternary ammonium salt, diethylaminoethyl (DEAE)-cellulose, a polymyxin B crosslinked polymer, polylysine, and a derivative thereof.

7. The method of claim 5, wherein the cationic polymer comprises cholestyramine, colestipol, colesevelam, or is the enteric-coated formulation thereof.

8. The method of claim 2, wherein an enteric-coated formulation of the cationic polymer is configured to sequestrate the PAMP produced by intestinal microorganisms, to alleviate related disease.

9. The method of claim 8, wherein the related disease caused by the PAMP is inflammatory diseases, metabolic syndrome, type-2 diabetes, obesity, tissue damage, alcohol related liver diseases, non-alcohol steatohepatitis, drug induced liver injury, fatty liver, cirrhosis, multiple tumors and cancers, organ failure, or sepsis.

10. The method of claim 2, wherein the cationic peptide is at least one selected from a group consisting of polylysine, α-defensin-5, α-defensin-6, and a structurally modified derivative thereof.

11. The method of claim 2, wherein the cationic peptide or the derivative thereof is configured to balance gut microbiota of the subject, to alleviate or treat related diseases.

12. The method of claim 5, wherein the enteric-coated formulation of the cationic peptide or the derivative thereof is applied to prevent or treat inflammatory related diseases which are derived in part by PAMP flowed via portal vein from the gut; and the inflammatory bowel diseases are Crohn's disease (CD) or ulcerative colitis (UC).

13. The method of claim 5, wherein the cationic polymer as active pharmaceutical ingredient is formulated in an enteric coated tablet or capsule for targeting release in specific section of an intestine.

14. The method of claim 1, wherein the PAMP produced by intestinal microorganisms comprises bacterial endotoxin, lipopolysaccharides, short-chain fatty acids, intestinal hydrogen sulfide, indole sulfate, short-chain fatty acids, DNA or RNA fragments, and bacterial flagellar proteins.
